(19) United States
(12) Patent Application Publication
(10) Pub. No.: US 2025/0064645 A1
(43) Pub. Date: Feb. 27, 2025

(54) IODINATED, INTELLIGENT WOUND DRESSING SYSTEM AND METHOD

(71) Applicant: InvictusHeal, Palo Alto, CA (US)

(72) Inventors: Eric LeVeen, Palo Alto, CA (US); Craig N. Lambert, San Jose, CA (US)

(21) Appl. No.: 18/814,178

(22) Filed: Aug. 23, 2024

Related U.S. Application Data
(60) Provisional application No. 63/534,244, filed on Aug. 23, 2023.

(51) Int. Cl. A61F 13/00; A61F 13/01; A61F 13/05

(52) U.S. Cl. CPC A61F 13/00063; A61F 13/01017; A61F 13/01029; A61F 13/01042; A61F 2013/0017; A61F 2013/00285; A61F 13/05

(57) ABSTRACT

A wound dressing system includes a control/monitor subsystem with a microprocessor configured for optimizing, wound healing based on wound condition parameters and medication concentrations. An intelligent, wound dressing includes: an inner, patient contact layer; and intermediate reservoir layer configured for retaining a quantity of medication; and an outer cover layer. The control/monitor subsystem can utilize artificial intelligence for optimizing, wound healing outcomes, and can interface with the wound dressing via a hardwired connection, a wireless (RF) connection or via the Internet (cloud).

### CROSS-REFERENCE TO RELATED APPLICATION

This application is a non-provisional of and claims priority in U.S. Patent Application Ser. No. 63/534,244, filed Aug. 23, 2023, which is incorporated herein by reference.

### BACKGROUND OF THE INVENTION

### 1. Field of the Invention

The present invention relates generally to wound dressing systems and methods, and in particular to an iodinated, intelligent, wound dressing system and method.

### 2. Description of the Related Art

The incidence of infection in wounds may depend upon the causative factors in the type and extent of damage to the skin and tissue wounds resulting from accidental causes and the presence of foreign bodies. Examples include wounds exposed to environmental contaminants outdoors, when a foreign body is present or when the blood supply is compromised. When wounds are denuded of overlying skin, there is an additional tendency towards infection from airborne contamination. When a significant amount of underlying tissue is exposed, a risk of infection may exist, even in clean environments such as operating rooms. One skilled in the art will understand that that the incidence of infection even in clean operating rooms may be proportional to the length of time the tissue is exposed to air in many instances. Antibiotics may need a blood supply since they are dependent on this to reach devitalized tissue to stop infection.

In certain instances, the healing of tissue in this state may have prolonged exposure to air. Drying of wounded tissue during long periods of exposure to air devitalizes it, lowering resistance of the contaminated tissue to infection. Another factor detrimental to the healing of open wounds may be the loss of carbon dioxide to the air from the wound surface. Such loss may produce a respiratory alkalosis of the local tissue leading to an alkaline shift in the pH. This alkaline shift in pH may result in an undesirable shift in the hemoglobin/oxyhemoglobin dissociation curve which, in turn, stabilizes the oxyhemoglobin and inhibits its conversion to reduced hemoglobin at reduced oxygen tensions. One skilled in the art will understand that wounds are desirably maintained to the relatively acid pH so that the oxygen tension is raised. In many examples, applying appropriate topical treatments may benefit the healing of a wound.

Various products have been developed, intended to discourage infection when topically applied. Antibiotics and preparations containing silver salts or sulfonamide compounds have been used with some success. However, some wounds that result from burns often become infected notwithstanding such preventative application. Various prior art wound dressings have been developed to address the need to prevent wound infection, but have not heretofore demonstrated the required effectiveness. Iodine has been used advantageously to treat wounds and prevent infection by virtue of its germicidal, algaecide, sporicidal, amoebicidal, fungicidal, virucidal and bactericidal properties. Its properties are most effective, however, in the treatment of a wound or burn when iodine contacts with the surface uninterrupted. This may require continuous and repeated application of the iodine.

It has been shown clinically that a flush of aqueous free iodine with only 2 ppm may be sufficient to prevent peritonitis in patients receiving peritoneal dialysis as a treatment for renal failure. An iodine saline flush has also proved curative in other cases of peritonitis not related to dialysis. Furthermore, an aqueous iodine solution of 2 ppm iodine is non-irritating, even when placed directly in the eye. A 1 ppm iodine solution however is insufficient to kill organisms and is the meager amount released from 10% PVP Polyvinylpyrrolidone.

An iodine solution may be preferential such is the reason Betadine was taken off the market when it was reported that methicillin resistant staphylococcus and pseudomonas were growing in the solutions used to prep skin at surgery. One skilled in the art will understand that the saline will wash away glucose, which would otherwise tend to convert free iodine to inactive iodine. Subsequent studies have indicated that in certain instances, rather than the two parts per million shown effective in saline solution, a higher concentration between five and 10 ppm of free iodine may be used when glucose and protein are not flushed away. In such a concentration, the iodine has been found to be bacterial, virucidal, fungicidal, and non-inflammatory, and non-irritating to tissue. Use of a lightly crosslinked polyurethane, which has been complexed with free iodine may be used to cause iodine to be released in therapeutic concentrations when exposed to an aqueous environment.

An open cell, polyurethane sponge can be effective in treating and preventing vaginitis when complexed with iodine and used as a tampon on the mucosa, and well known for its effectiveness in preventing infection on undamaged external tissue, but the prior art does not teach nor advise the use of such an iodophor in direct contact with an open wound or burn. In various embodiments of the invention, a wound dressing is described that can protect an open wound or burned tissue from direct exposure to air, and can maintain the necessary aqueous environment, tissue needs for hydration but also can the release medicants, such as iodine into the wounded tissue with which it is in contact. Iodine, in some embodiments is configured and used as a surgical drape, that in addition to providing a barrier against direct exposure to air, such a dressing contains iodine in its adhesive, rendering it bactericidal to the skin. This prevents infection of the wound from airborne contaminants in the operating room and the battlefield but also the commensal organisms populating our skin.

Heretofore there has not been available a system or method for iodinated, intelligent, wound dressing with the advantages and features of the present invention.

### BRIEF SUMMARY OF THE INVENTION

The present invention generally provides an iodinated, intelligent, wound dressing system and method. Various embodiments of the invention provide a dressing which overcomes the drawbacks of prior art. While the wound dressing protects wounds, including those resulting from burns, from infection, on the outside, it is occlusive to prevent loss of CO2 from the surface of the wound. Only by preventing the loss of carbon dioxide from the wound surface can the acid pH of the wound be maintained. Oxygen is not released from oxyhemaglobin in an alkaline pH. The film on the back of the dressing by being occlusive adds to the other embodiments to create the optimal environment for wound healing. The wound dressing, which may be hydrophilic, is capable of releasing therapeutic amounts of free iodine to the wound over prolonged periods in accordance with various embodiments of the invention. In certain examples, the wound dressing may be replenished when medicants contained therein fall below a threshold and, in certain instances, may be replenished without removal from contact with the wound or during shortened period of time during which the dressing is moved. In short, embodiments of the invention provide a wound dressing comprised of a hydrophilic, but insoluble material advantageously a polymer, a compound capable of being reversibly complexed, with elemental iodine, thereby permitting the release of therapeutic amounts of free iodine into a wound which is brought into contact with the dressing. Finally the embodiment of being occlusive preserves the other elements to make this dressing unique. One skilled in the art will understand that other types of material that have similar characteristics to those described above may also be used in other embodiments of the invention.

### BRIEF DESCRIPTION OF THE DRAWINGS

The drawings constitute a part of this specification and include exemplary embodiments of the present invention illustrating various objects and features thereof.

FIG. 1 is a schematic block diagram of an intelligent wound dressing system embodying an aspect of the present invention.

FIG. 2 is an exploded, perspective view of a multi-layer dressing embodying an aspect of the present invention.

FIG. 3a is a perspective view of the dressing.

FIG. 3b is a plan view of the dressing.

FIG. 3c is an enlarged, perspective view of a silicon inlet/outlet (I/O) port.

FIG. 4a is a perspective view of a wound drape embodying an aspect of the present invention.

FIG. 4b is a cross-sectional view of the wound drape.

FIG. 5a is a plan view of a pressure-driven, flow-through dressing embodying an aspect of the present invention.

FIG. 5b is a cross-sectional view thereof.

FIG. 6 is a cross-sectional view showing a pair of counter-rotating rollers configured for calendaring a polyurethane foam dressing with an iodine solution.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Said terminology will include the words specifically mentioned, derivatives thereof and words of similar meaning.

### II. Preferred Embodiments

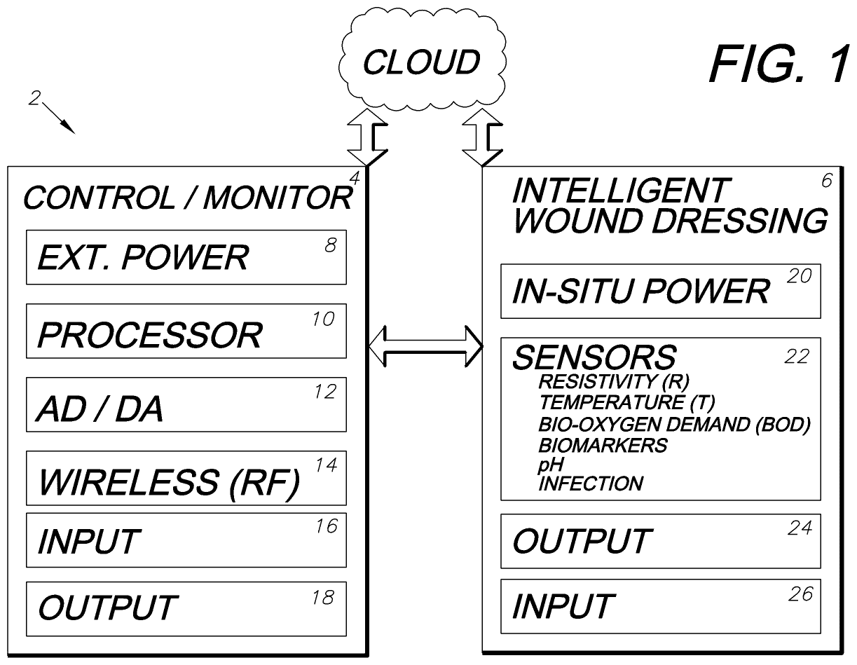

Referring to the drawings in more detail, FIG. 1 shows an iodinated, intelligent, triple-layer wound dressing system 2 embodying an aspect of the present invention. A control/monitor subsystem 4 is connected to an intelligent, wound dressing 6.

The control/monitor subsystem 4 can include a power source 8, which can comprise, for example, 120V alternating-current (AC), direct-current (DC) from batteries or a generator, etc. The control/monitor subsystem 4 further includes: a processor (CPU) 10; an analog-digital (AD) and digital-analog (DA) converter 12; a wireless (RF) interconnect device 14; an input component 16; and an output component 18.

Also as shown in FIG. 1, the intelligent wound dressing 6 can include an in-situ power component 20 and a sensor array 22 for various wound parameters, e.g., without limitation, resistivity (R), temperature (T), bio-oxygen demand (BOD), biomarkers, pH and infection levels. The wound dressing includes an output component 24 and an input component 26.

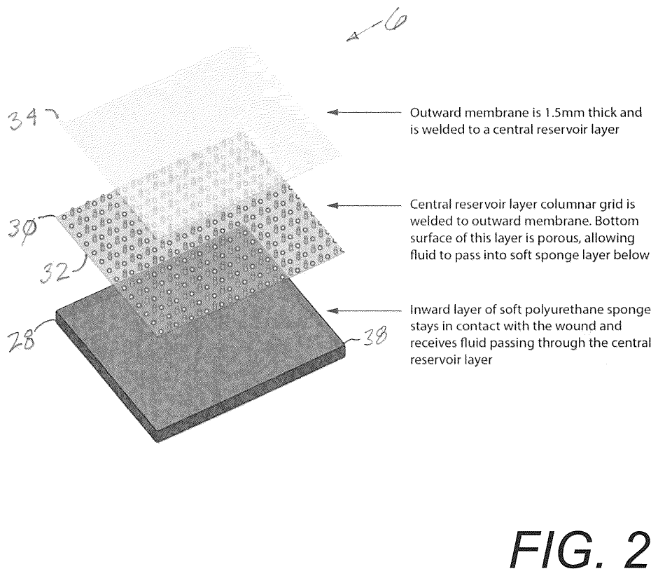

As shown in FIG. 2, the dressing 6 includes an inner, proximal, wound-contact sponge layer 28, which can comprise, without limitation, a soft, porous, polyurethane material. A central reservoir layer 30 has a porous bottom surface 32, which enables fluid to pass through from the proximal, inner sponge layer 28 and into the central layer 30, which has a columnar grid configuration. An outer, distal membrane layer 34 can comprise, without limitation, 1.5 mm thick polyurethane film, and preferably has a relatively high tensile strength. The membrane layer 34 preferably has sufficient tensile strength to hold a tourniquet in place, which can be secured to itself with staples.

An aspect of the intelligent dressing 6 enables activating the central reservoir layer 30 between the outer, distal membrane layer 34 and the inner sponge layer 28, which stays in contact with the wound. The central reservoir layer 30 functions as a retention chamber between the inner and outer layers 28, 34.

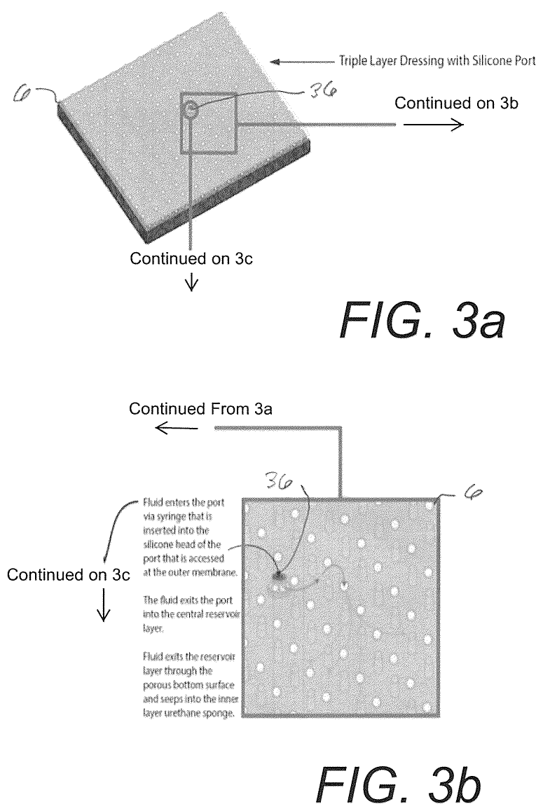

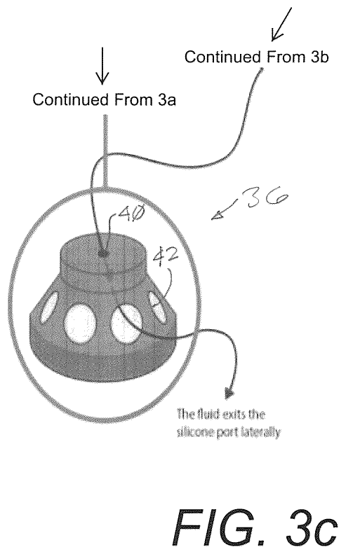

As shown in FIGS. 3a-3c, the dressing 2 can be provided with a silicone access port 36, which can be configured for receiving injections of fluids, such as aqueous iodine and other medicaments via a syringe. The injected fluids can fill the central, column-gridded reservoir layer 6, and are applied to the patient's wound via a porous film layer 38 on the inner sponge layer 28. When more fluid is required to flood the sponge layer 28 for dispersion to the skin, the access port 36 can be “recharged” as necessary, thus providing a continuous and rechargeable dressing. It will be appreciated that recharging the central reservoir layer enables extending the bactericidal action on the wound for extended periods (e.g., several days) without changing the dressing 6.

FIG. 3a shows the access port 36 approximately centrally located on the dressing 2. FIG. 3b shows the access port 36 located near a perimeter of the wound dressing 6. FIG. 3c is an enlarged view of the access port 36 showing an injection point 40 at the top and lateral exit ports 42.

Access port location can be chosen based on wound characteristics and desired fluid flow mechanics, for example, gravitational or under negative pressure (suction). The dressing 6 can be tilted from horizontal to facilitate gravity flow. Moreover, multiple access ports 36 can be placed as desired on the dressing 2. When the wound re-epithelializes, appropriate adjustments can be made to the treatment protocol, including iodine levels, recharging frequency, etc.

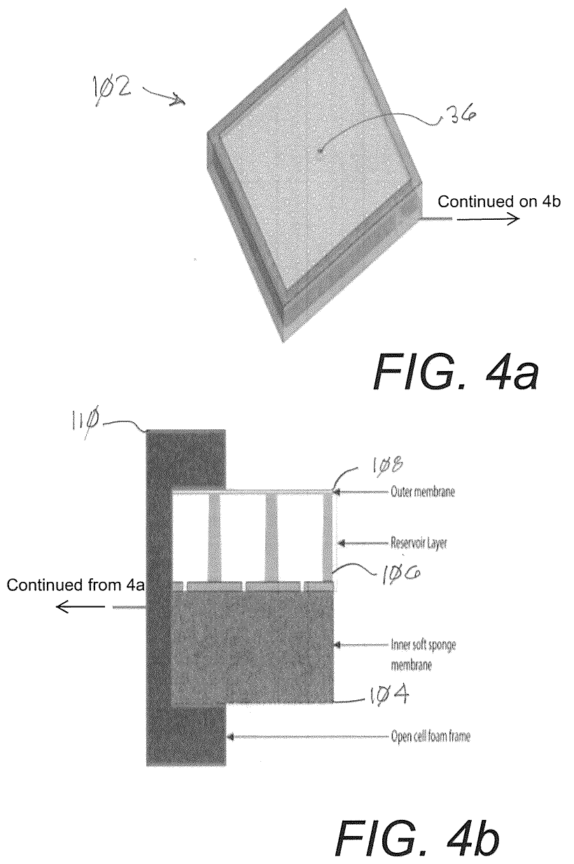

FIGS. 4a-4b show a modified embodiment of the invention comprising a dressing 102 with an inner, proximal, soft sponge membrane layer 104 configured for wound surface engagement, a reservoir layer 106 and an outer membrane layer 108, which can comprise a polyurethane film as well as an iodinated urethane adhesive for blocking airborne contaminants, covering operations and partial thickness cuts to the skin, e.g., for skin graft procedures. The dressing 102 components can be reversibly complexed with elemental iodine. When attaching a port or open cell drain to a Wound VAC® negative pressure wound therapy (NPWT) system, the wound can be aspirated. Such NPWT equipment, components and supplies are available from 3M® of Minneapolis, Minnesota.

As shown in FIG. 4b, an open cell foam frame 110 can be formed around the inner sponge layer 104, the intermediate reservoir layer 106 and the outer membrane layer 108 at their respective perimeters.

The manufacture of iodinated polyurethane foams can be achieved in accordance with various embodiments of the invention. For example, polyurethane (PU) foams can be treated as porous media materials. Darcy's law of fluid mechanics (flow) defines the flux (Q) of a liquid through a porous material as:

$$Q = - ( k / μ T ) dP$$

Where k is permeability, p is the liquid's dynamic viscosity, T is the thickness of the foam and dP is the applied pressure drop. The polyurethane (PU) foams can be medical grade, chosen for their strong affinity to iodine, resulting in a strong physical absorption through the creation of PU-iodine charge-transfer complexes the benzene rings of the PU foam. The iodinated PU foams may retain iodine for long periods of time, while producing a sustained outward flux of this antimicrobial molecule to wounds in contact with the foam.

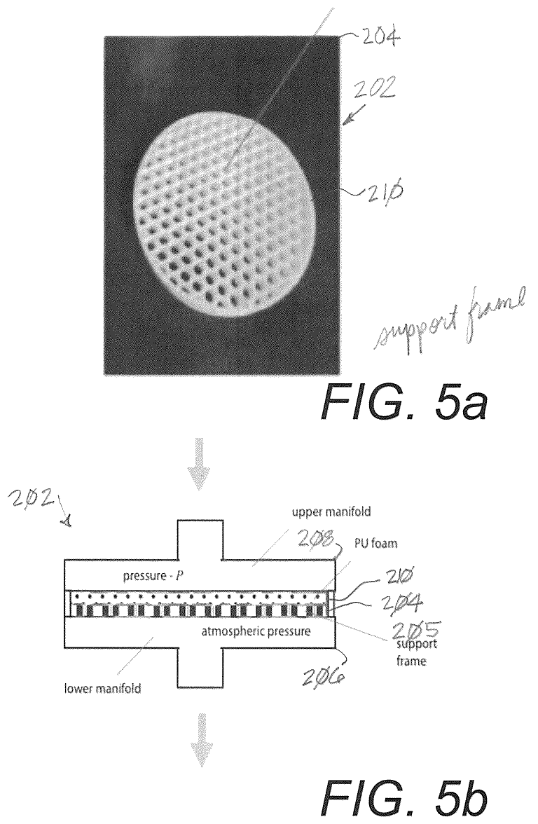

FIGS. 5a-5b show another modified or alternative embodiment iodinated intelligent, wound dressing, 202 with a pressure-driven, flow-through polyurethane foam component 204 with a support frame 205, lower and upper manifolds 206, 208 and a polyurethane foam reservoir layer 210.

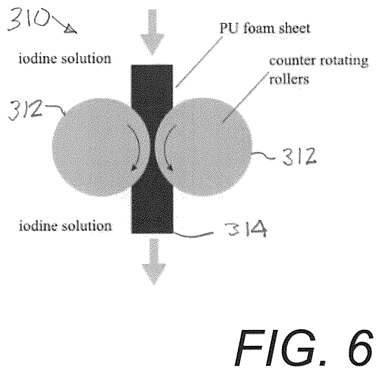

FIG. 6 shows a dressing calendaring device 310 with counter-rotating rollers 312 configured for achieving a specified iodine concentration in a polyurethane foam sheet 314. For example, and without limitation, 1.6% of a standard, 2% iodine tincture solution can be utilized with uncompressed polyurethane foam sheets 314 with thicknesses of approximately 6 mm and gaps between the rollers 312 of approximately 2 mm. Upon exiting the rollers 312, the compressed foam sheet 314 will expand within the iodine solution and through the interplay of elasticity, capillary forces in the ferocity of the flow, the iodine solution will enter the boat by an amount that can be determined with a mass balance calculation. Such processing methods have achieved relatively uniform concentrations of iodine within the folds. Samples can be rinsed and squeeze spell most of the residual water. Such samples can then be desiccated for approximately 2 hours at temperatures of approximately 58° C. for a week pursuit of iodine in the polyurethane foams of approximately 5 mg/liter of I2.

Various strains of bacteria are known for their resistance to other forms of dressings, including Staphylococcus aureus, and Pseudomonas aeruginosa, and may be used to test samples. Such species are frequently found in wound infections, making them suitable targets for testing processes. The evaluation of samples may include measuring the rate of bacterial growth, the sum of inhibition around the dressing and the survival rate of bacteria.

The iodinated intelligent wound dressing systems of the present invention can utilize artificial intelligence for optimizing outcomes. Moreover, they can be adapted to different environments, various medications, patient interface (dressing) materials; wound configurations, available equipment and patient conditions. Still further, various component configurations can be utilized or practicing the present invention. For example, the access ports can be configured with valves having both open and closed positions. Alternatively, the access ports can be configured for one-way injection or administration of medications.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. An iodinated, intelligent, wound dressing system, which comprises:
a control/monitor subsystem including a microprocessor with input and an output;
said microprocessor configured for programming to optimize application of medication to wounds;
a dressing including: a first, inner layer configured for application directly to a patient wound; an intermediate reservoir layer configured for containing a quantity of a medication; and an outer membrane configured for covering said reservoir layer;
a sensor connected to said dressing and configured for sensing a condition of said dressing; and
a user interface connected to said microprocessor and configured for alerting the user to a wound condition parameter.

2. The dressing system of claim 1, wherein said inner layer comprises a porous polyurethane material.

3. The dressing system of claim 2 wherein said intermediate reservoir layer comprises gridded configuration.

4. The dressing system of claim 3 wherein said intermediate reservoir layer comprises a collimated construction configured for retaining a volume of medication.

5. The dressing system of claim 4, wherein said outer membrane layer comprises a polyurethane film.

6. The dressing system of claim 5, which includes:
an access port mounted on said outer membrane layer and configured for receiving a quantity of medication and transferring same to said intermediate reservoir layer.

7. The dressing system of claim 6, wherein said access port includes a base mounted on said outer membrane layer, an injection point configured for receiving an injection of medication from a syringe and multiple lateral fluid discharge ports between said injection point and said base.

8. The dressing system of claim 1, which includes:
said wound dressing having a perimeter; and
a frame comprising an open cell foam material mounted on said wound dressing perimeter.

9. The dressing system of claim 1, which includes:
a lower manifold between said central layer and said inner layer, said lower manifold configured for dispersing medication to said wound surface through said inner layer; and
an upper manifold mounted on and fluidically communicating with said central layer; and
said manifolds comprising a porous, polyurethane foam material.

10. The dressing system of claim 1 wherein said access port includes a valve with open and close configurations.

11. The dressing system of claim 1 wherein said inner layer includes a porous film material.

12. The dressing system of claim 1, wherein said control/monitor subsystem is connected to said intelligent dressing via one of:
a hardwired connection;
a wireless (RF) connection; and
a connection via the Internet (cloud).

13. A wound treatment method, which comprises the steps of:
applying a multilayer dressing to a surface wound;
providing a multi-layer dressing with: an inner layer comprising a polyurethane sponge material; an intermediate reservoir layer with a polymer grid and a porous inner surface connected to said inner layer; and an outer membrane comprising a polyurethane material applied to said intermediate layer;
providing access port on said outer layer for supplying a liquid medication to said intermediate reservoir layer;
saturating said intermediate reservoir layer with a liquid medication;
placing said dressing on patient in covering relation over a topical wound; and
monitoring said medication level and patient healing parameters.

14. The wound treatment method according to claim 13, which includes the additional step of: providing an iodine solution for said medication.

15. The wound treatment method according to claim 13 wherein said iodine solution is in the range of 0.5%-3%.

16. The wound treatment method according to claim 13 wherein:
said wound dressing elutes iodinated medication in the range of 2 ppm-15 ppm; and
the patient receives medication in the range of 1-2 mg daily.

17. The wound treatment method according to claim 13, which includes the additional step of metering solution into said dressing.

18. The wound treatment method according to claim 13, which includes the additional step of providing a calendaring device configured for optimizing the medication content in said dressing by passing said dressing through a pair of rollers on said calendaring device.

19. The wound treatment method according to claim 13, which includes the additional step of configuring said access port for selective closure with a valve open and closed configurations.

20. The wound treatment method according to claim 13 wherein said control/monitoring subsystem utilizes artificial intelligence for optimizing patient outcomes.
